(19) United States
(12) Patent Application Publication
(10) Pub. No.: US 2009/0023994 A1
(43) Pub. Date: Jan. 22, 2009

(54) Endoscope Insertion Portion and Endoscope

(76) Inventors: Yasuhito Kura, Tokyo (JP); Katsutaka Adachi, Tokyo (JP)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/918,019

(22) PCT Filed: Mar. 15, 2006
(86) PCT No.: PCT/JP2006/305129

§ 371 Date: Oct. 5, 2007

(30) Foreign Application Priority Data
Apr. 5, 2005 (JP) 2005-109095
Apr. 5, 2005 (JP) 2005-109097

(51) Int. Cl. A61B 1/00

(52) U.S. Cl. 600/114

(57) ABSTRACT

In order to realize an endoscope insertion portion and an endoscope system easily inserted into the body cavity such as a large intestine and is capable of improvement of invertibility into the body cavity without imposing pain on a patient, an endoscope insertion portion of the present invention includes an endoscope insertion portion capable of being inserted into a subject; a spiral tube as a propulsive force generation section mounted on the outer periphery side of the endoscope insertion portion and rotating around the longitudinal axis of the endoscope insertion portion; and a friction reduction spiral portion provided at a predetermined position on the outer periphery of a spiral-formed portion, for example, on the proximal end side, the friction reduction spiral portion being provided as a frictional force reduction member mounted on the spiral tube, reducing a frictional force generated between the spiral tube and a body-cavity inner wall.

### TECHNICAL FIELD

The present invention relates to endoscope insertion portions and endoscope systems and, in particularly, to an endoscope insertion portion and an endoscope system for introduction into a body cavity.

### BACKGROUND ART

Conventionally, endoscopes have been widely employed in the medical-application field. Such an endoscope can observe an affected area or the like in a body cavity by inserting an endoscope insertion portion into the body cavity and, as needed, perform treatments and procedures by inserting a treatment instrument into a forceps channel.

Generally, the endoscope insertion portion is formed with a bending portion on a distal end side. The bending portion includes a plurality of bending pieces. The bending portion is bent, for example, vertically/horizontally by towing an operation wire connected with the bending pieces. The towing of the operation wire is performed by an operator's turning, for example, a bending knob provided at an operation portion.

An operator, when inserting the endoscope insertion portion into a complicated body cavity, for example, a tube cavity drawing a 360° loop like a large intestine, bends the bending portion by operating the bending knob and inserts the distal end portion of the endoscope insertion portion toward a portion to be observed while twisting the insertion portion. However, such operation of an endoscope requires a skill to a degree that the insertion portion can be smoothly inserted up to the deep region of the complicated large intestine in a short time.

An inexperienced operator may lose track of an insertion direction while inserting an insertion portion to a deep region, which may cause the operator to confront insertion difficulty or largely deviate running state of an intestine from a target route. Accordingly, there have conventionally been a variety of proposals for enhancing the insert ability of the endoscope insertion portion. For example, Japanese Patent Laid-Open Publication No. Hie 10-111996, hereinafter referred to as “Patent Document 1”, has proposed a propulsion system for medical instrument capable of guiding a medical instrument easily and with a slight invasion up to the deep region of a living tube. The propulsion system has a rotating member formed with a rib provided diagonally to an axial direction of the rotating member as a propulsive force generation section. Accordingly, the propulsion system in Patent Document 1 described above rotates the rotating member, so that the torque of the rotating member is converted into a propulsive force by the rib and the medical instrument connected to the propulsion system is moved toward the depth direction by the propulsive force. This permits the propulsion system in Patent Document 1 described above to insert the medical instrument into a body cavity with a slight invasion and without giving a physical burden to a patient.

However, for the propulsion system for medical instrument described in Patent Document 1, it is difficult to insert into a bending body cavity such as a large intestine while the internal large intestine is being observed. Accordingly, the above-described propulsion system for medical instrument has difficulty in inserting a medical instrument into a large intestine because a rotating member used in the medical instrument is made to abut against a bending portion of the large intestine when the medical instrument reaches the bending portion of the large intestine.

In this case, an operator may make a determination for appropriate treatment too late. Moreover, for example, an operator may continue to rotate a rotating member used in a medical instrument although the distal end portion of the medical instrument has reached the proximity of a caucus portion. In addition, after inserting the medical instrument into the body cavity such as a large intestine, an operator may take an action for inserting an endoscope insertion portion along the instrument into the large intestine, resulting in double labor and troublesome.

In view of the above-described problems, it is an object of the present invention to provide an endoscope insertion portion and an endoscope system capable of easy insertion of the endoscope insertion portion into a body cavity such as a large intestine and improvement in the endoscope insertion portion to be inserted into the body cavity without giving a pain to the patient.

### DISCLOSURE OF INVENTION

### Means for Solving the Problem

A first endoscope insertion portion according to the present invention comprises: an insertion portion capable of being inserted into a subject; a propulsive force generation section mounted on an outer-peripheral surface of the insertion portion and rotating around a longitudinal axis of the insertion portion; and frictional force reduction means mounted on the propulsive force generation section and reducing a frictional force generated between the propulsive force generation section and a body-cavity inner wall.

A second endoscope insertion portion according to the present invention comprises: an insertion portion capable of being inserted into a body to be inspected; a propulsive force generation section mounted on an outer-peripheral surface of the insertion portion and rotating around a longitudinal axis of the insertion portion; and propulsive force reduction means mounted on the propulsive force generation section and reducing a propulsive force generated by the propulsive force generation section.

A first endoscope system according to the present invention comprises: a slender and flexible insertion portion; a flexible insertion-portion guide portion mounted on the outer periphery side of the insertion portion and formed with, on an outer-periphery surface, a spiral-shaped portion generating a propulsive force by rotation in contact with a body-cavity inner wall; a guide portion rotation device rotating the insertion-portion guide portion around a longitudinal axis in a predetermined direction; and frictional force reduction means mounted on the insertion-portion guide portion and reducing a frictional force against the body-cavity inner wall.

A second endoscope system according to the present invention comprises: a slender and flexible insertion portion; a flexible insertion-portion guide portion mounted on the outer periphery side of the insertion portion and formed with, on an outer-periphery surface, a spiral-shaped portion generating a propulsive force by rotation in contact with a body-cavity inner wall; a guide portion rotation device rotating the insertion-portion guide portion around a longitudinal axis in a predetermined direction; and propulsive force reduction means mounted on the insertion-portion guide portion and reducing a propulsive force of the insertion-portion guide portion.

### BRIEF DESCRIPTION OF THE DRAWINGS

FIG. 1 is a view showing an overall configuration of an endoscope system in a first embodiment according to the present invention;

FIG. 2 is an external view showing the proximity of a distal end portion of an introductory tube in FIG. 1;

FIG. 3 is a descriptive view showing the introductory tube and an endoscope in FIG. 1;

FIG. 4 is a sectional view taken on line A-A in FIG. 3;

FIG. 5 is a descriptive view showing a configuration of a rotation mechanism portion;

FIG. 6 is a descriptive view showing the configurational property of a spiral-shaped portion;

FIG. 7 is an external view of a spiral tube in FIG. 3;

FIG. 8 is a descriptive sectional view showing a configuration of the spiral tube in FIG. 7;

FIG. 9 is a descriptive view showing such a state that the introductory tube with an insertion portion inserted therein is inserted from an anus;

FIG. 10 is a descriptive view showing such a state that a distal end body of the introductory tube with an insertion portion inserted therein is inserted up to the proximity of a caucus portion;

FIG. 11 is a view showing an overall configuration of an endoscope system in a second embodiment according to the present invention;

FIG. 12 is a descriptive view showing connection of an endoscope insertion portion with an endoscope rotation device (referred to as a “rotation device”) in FIG. 11;

FIG. 13 is a partially sectional view with the endoscope insertion portion in FIG. 12 cut in a longitudinal direction;

FIG. 14 is an external view of the introductory tube (guide tube) in FIG. 13;

FIG. 15 is a descriptive view showing a configuration of the introductory tube (guide tube) in FIG. 14;

FIG. 16 is a descriptive view showing the configurational property of a spiral-shaped portion;

FIG. 17 is a descriptive view of an endoscope insertion portion inserted into a large intestine;

FIG. 18 is a descriptive view showing such a state that an endoscope insertion portion is inserted while an S-shaped colon portion is being drawn into an α-loop shape;

FIG. 19 is a descriptive view of an endoscope insertion portion inserted into a deep region of an intestine;

FIG. 20 is an external view of the introductory tube (guide tube) showing a deformed example of FIG. 14;

FIG. 21 is an external view of an introductory tube (guide tube) configured by attaching covering tape at a predetermined position as covering means;

FIG. 22 is an external view of an introductory tube (guide tube) configuring an endoscope system in a third embodiment according to the present invention; and

FIG. 23 is an external view of the introductory tube (guide tube) showing a deformed example of FIG. 22.

### BEST MODE FOR CARRYING OUT THE INVENTION

The present invention will now be described in detail with reference to the drawings showing embodiments thereof.

### First Embodiment

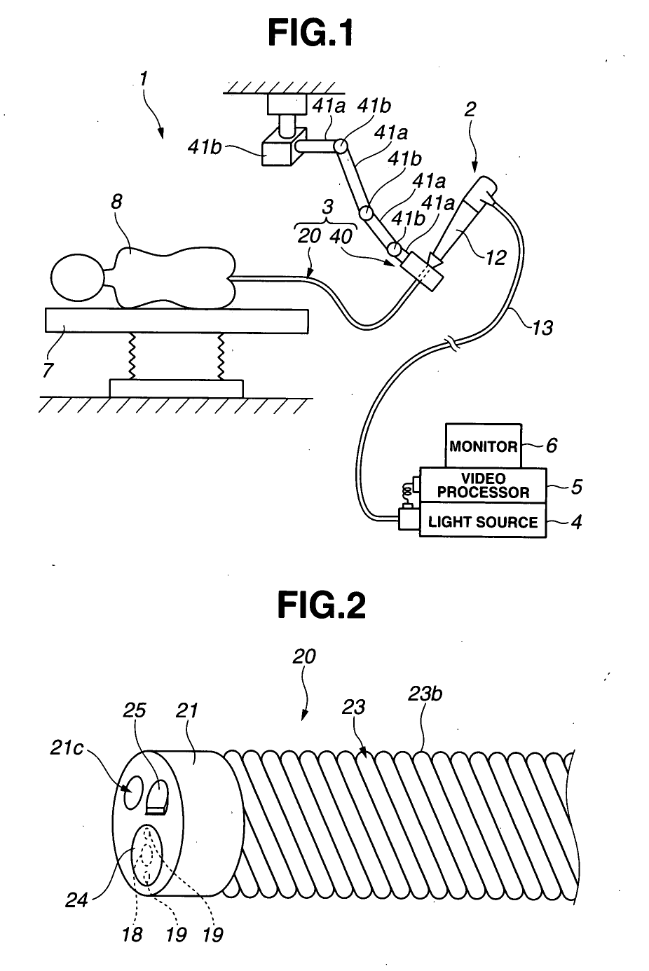

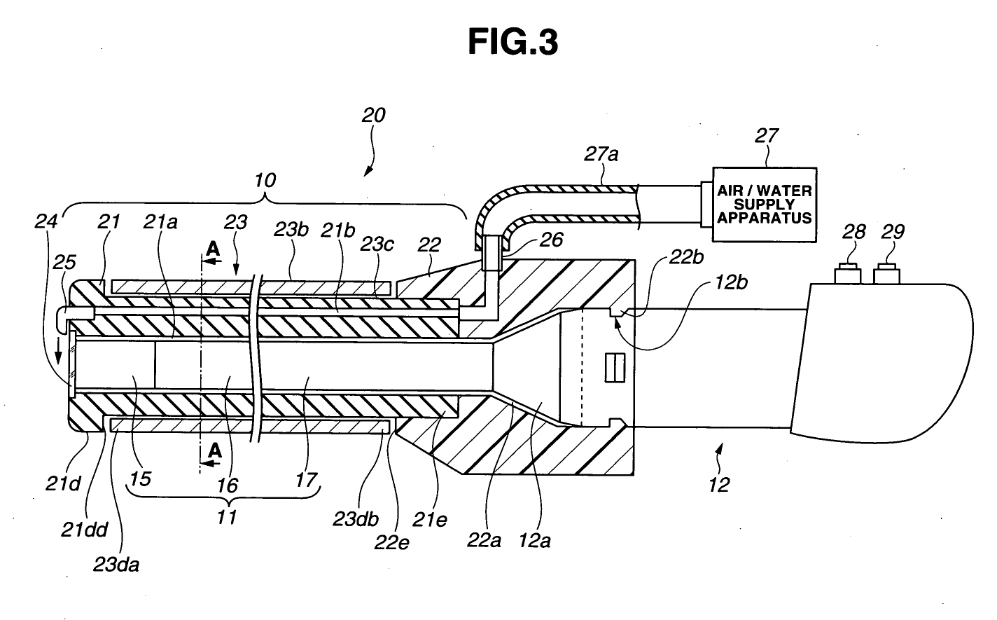

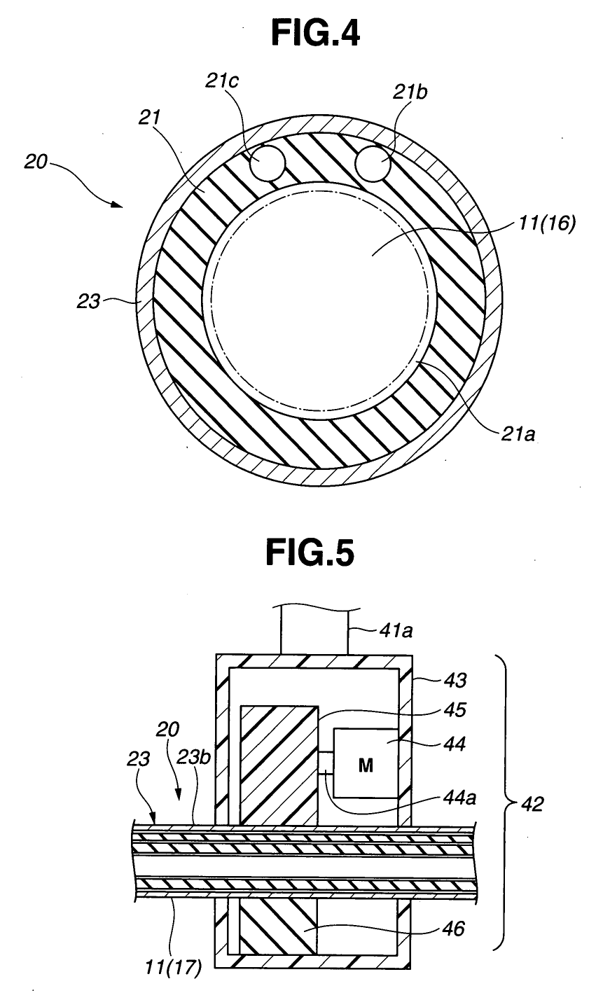

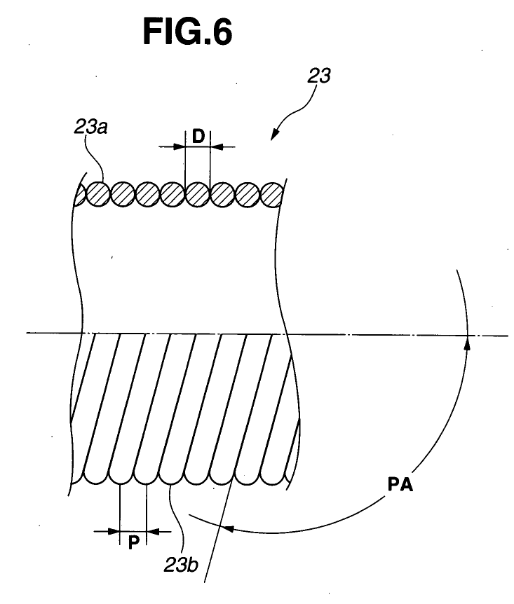

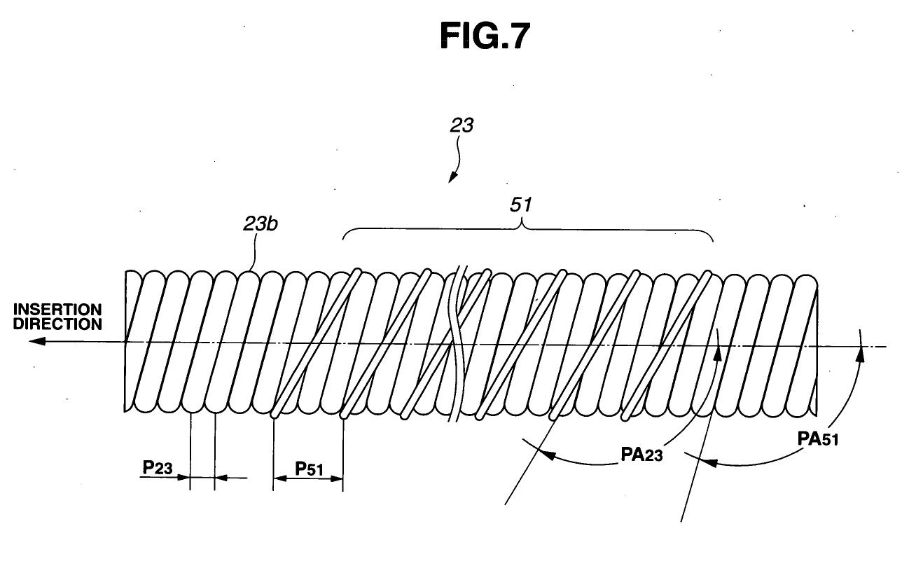

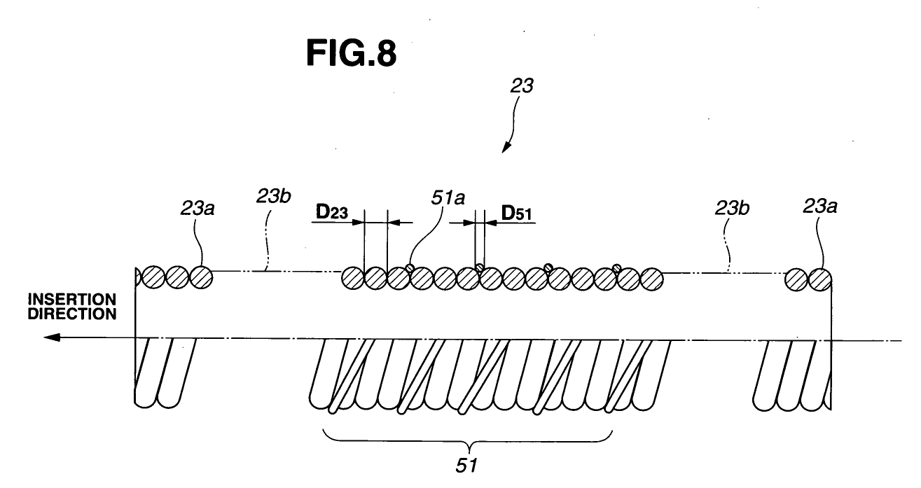

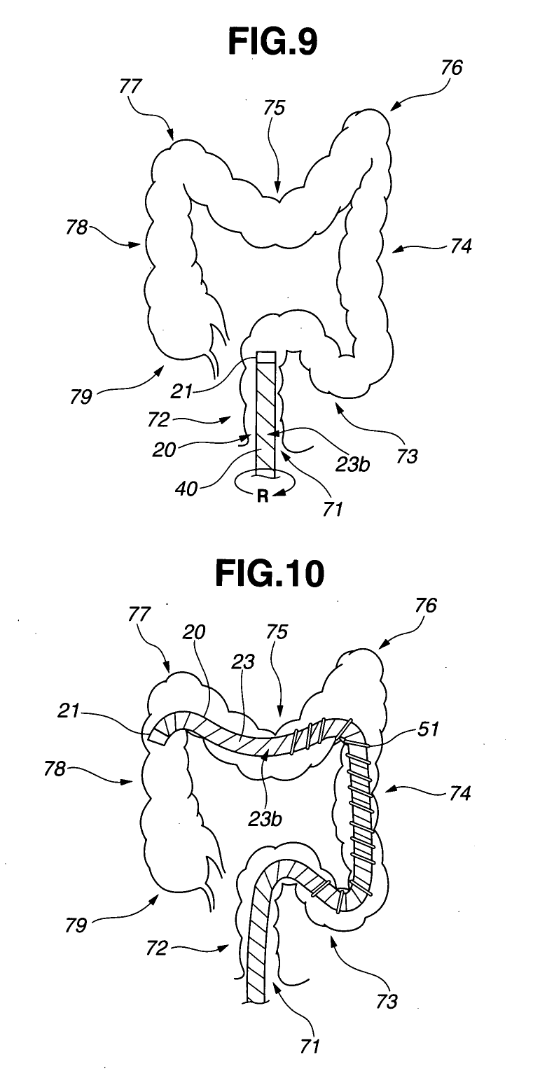

FIGS. 1 to 10 are views of an endoscope insertion portion and an endoscope system according to the present invention. FIG. 1 is a view showing an overall configuration of an endoscope system in a first embodiment, FIG. 2 is an external view showing the proximity of a distal end portion of an introductory tube (guide tube) in FIG. 1, FIG. 3 is a descriptive view showing the introductory tube and an endoscope in FIG. 1, FIG. 4 is a sectional view taken on line A-A in FIG. 3, FIG. 5 is a descriptive view showing a configuration of a rotation mechanism portion, FIG. 6 is a descriptive view showing the configurational property of a spiral-shaped portion, FIG. 7 is an external view of a spiral tube in FIG. 3, FIG. 8 is a descriptive sectional view showing a configuration of the spiral tube in FIG. 7, FIG. 9 is a descriptive view showing such a state that the introductory tube with an insertion portion inserted therein is inserted from an anus, and FIG. 10 is a descriptive view showing such a state that a distal end body of the introductory tube with an insertion portion inserted therein is inserted up to the proximity of a caucus portion.

As shown in FIG. 1, an endoscope system 1 in the present embodiment comprises an endoscope 2 and an endoscope insertion assisting tool 3. The endoscope 2 is provided with a light source 4, a video processor 5 and a monitor 6 as external units. The light source 4 supplies illuminating light to the endoscope 2. The video processor 5 has a control circuit for performing various types of control and a signal processing circuit, and transmits a driving signal for driving an image pickup device (not shown) provided to the endoscope 2, converts an electric signal transmitted after photoelectric transfer by the image pickup device into a video signal and outputs the video signal to the monitor 6. The monitor 6, after receiving the video signal outputted from the video processor 5, displays an endoscope image on its screen.

The endoscope 2 comprises a flexible slender endoscope insertion portion 11 as an endoscope insertion portion and an operation portion 12 attached onto the proximal end portion of the endoscope insertion portion 11. The endoscope 2 has a universal cord 13 extending from the side portion of operation portion 12.

The endoscope insertion assisting tool 3 with the endoscope insertion portion 11 inserted therein comprises an introductory tube 20 as an insertion portion for guiding the endoscope insertion portion 11 in a depth direction of a body cavity and a rotation device 40 for rotating a spiral tube 23 described later of the introductory tube 20.

The rotation device 40 mainly comprises, for example, an arm portion 41 one end of which is attached onto a ceiling of an inspection room and a rotation mechanism portion 42 mounted on the other end of the arm portion 41. The arm portion 41 comprises a plurality of arm members 41a, for example, having different lengths and a plurality of joint portions 41b pivotally connecting the arm members 41a adjacent to each other. This permits a user to move the rotation mechanism portion 42 to a predetermined position with a slight effort. A detailed configuration of the rotation mechanism portion 42 will be described later.

As shown in FIGS. 2 to 4, the introductory tube 20 comprises an insertion portion cover 10 constituted of an observation window member 24 and an elastic cover tube 21, a proximal-end-side component 22 provided in a linked manner to the insertion portion cover 10 and a spiral tube 23 disposed on the outer periphery side of the insertion portion cover 10 and serving as a propulsive force generation section for generating a propulsive force at the introductory tube 20. In other words, the introductory tube 20 as an insertion portion is formed with the spiral tube 23 attached onto the outer-periphery surface side of the introductory tube 20 and serving as a propulsive force generation section rotating around a longitudinal axis of the introductory tube 20.

The elastic cover tube 21 is constituted of an elongated tubular member with low frictional resistance, made of, for example, Fluor ethylene resin such as PTFE (tetrafluoroethylene resin). The elastic cover tube 21 has a through hole 21 a with the endoscope insertion portion 11 inserted therein, which is axially drilled therethrough. Moreover, the elastic cover tube 21 has a through hole 21b as an air/water supply channel, axially drilled therethrough. Furthermore, the elastic cover tube 21 has a through hole 21c axially drilled therethrough as a treatment instrument insertion channel or suction channel as shown in FIG. 4.

In the front of the elastic cover tube 21 on the distal-end-portion side, the observation window member 24 is disposed at an opening of a through hole 21a on the distal end side together integrally with the elastic cover tube 21 by means of adhesion or the like. The proximal end side of the through hole 21 a is communicated with a through hole 22a described later, which is formed at the proximal-end-side component 22.

The observation window member 24 is formed of a transparent resin material with optical properties, such as polycarbonate. When the endoscope insertion portion 11 is inserted into the through hole 21 a, the front of a distal end portion 15 constituting part of the endoscope insertion portion 11 is made to abut on the inner-side surface of the observation window member 24. The observation window member 24 watertightly blocks the opening in the front surface of the elastic cover tube 21 and serves as an observation window for the endoscope 2.

One end side of the through hole 21b is communicated with an air/water supply nozzle 25 disposed near the distal end portion of the elastic cover tube 21. The opening of the air/water supply nozzle 25 faces the observation window member 24. On the other end of the through hole 21b, there is formed a mouth ring 26 so as to protrude to the outer periphery of the proximal-end-side component 22. One end of an air/water supply tube 27a is coupled to the mouth ring 26. On the other end side of the air/water supply tube 27a, an air/water supply apparatus 27 is provided in a linked manner. The air/water supply apparatus 27 is configured so as to perform driving control with a push-button switch 28 for air/water supply.

Accordingly, if the observation window member 24 has, for example, any filth on it, a user arbitrarily drives the air/water supply apparatus 27 to spray predetermined fluid such as air or liquid, for example, water as indicated by an arrow from the opening of the air/water supply nozzle 25 via the through hole 21b, thus washing away the filth adhering to the observation window member 24. By spraying, for example, air from the opening of the air/water supply nozzle 25, water droplets adhering to a surface of the observation window member 24 can be removed.

The through hole 21c is communicated with a channel opening portion formed at a predetermined portion of the proximal-end-side component 22. In using the through hole 21c as a treatment instrument insertion channel, a user inserts a treatment instrument such as a biopsy needle or a biopsy forceps into the channel opening portion. The treatment instrument is inserted into the through hole 21c as the treatment instrument channel and is exposed from an opening at a distal end portion of elastic cover tube 21 to outside. This permits a user to perform predetermined treatment. In using the through hole 21c as a suction channel, a channel opening has one end of a tube line connection member and the other end of the tube line connection member is connected, for example, to a suction tube line (not shown) extended from a predetermined suction device (not shown). The suction device is configured so as to perform driving control with a suction push-button switch 29.

Accordingly, the endoscope 2 inserted in the through hole 21 a comprises only an observation window 18 constituting an observation optical system and an illumination window 19 constituting an illumination optical system provided on a distal end surface of the endoscope insertion portion 11, thus minimizing the diameter of the insertion portion.

The spiral tube 23 is made of, for example, stainless and is formed by winding metal strand of predetermined diameter in a spiral manner so as to have predetermined flexibility. On an outer surface of the spiral tube 23, a spiral-shaped portion 23b is formed of a surface of metal strand.

The spiral tube 23 is constituted by forming a clearance 23c between an inner-peripheral surface of the spiral-shaped portion 23b and an outer-peripheral surface of the elastic cover tube 21 and covering an outer-peripheral surface of the elastic cover tube 21 and is rotatably disposed in a peripheral direction (around its axis) relative to an outer-peripheral surface of the elastic cover tube 21.

The spiral tube 23 is configured so as to rotate in a peripheral direction (around its axis) with the rotation mechanism portion 42 of the rotation device 40 as described later. The spiral tube 23 is not limited to a one-line configuration. For example, a spiral tube wound in a plurality of rows such as two-line and four-line may be used.

The spiral tube 23 can adjust propulsive force and traveling speed by changing a close contact between metal strands or variously setting spiral angles in forming the spiral tube by winding the metal strand in a spiral manner. Preferably, the metal strand 23a is formed by winding the metal strand in a left-handed spiral manner from its distal end toward its proximal end, thus improving insertability of the endoscope insertion portion 11 into a large intestine.

At a distal end portion of an outer-peripheral surface of the elastic cover tube 21, there is formed a protrusion portion 21 d for preventing the spiral tube 23 from dropping off. The spiral tube 23 is configured so that its front end portion 23da is made to abut against and latched by a rear face portion 21dd of the protrusion portion 21d, thus regulating forward movement of the spiral tube 23, while its rear end portion 23db is made to abut against and latched by a front face portion 22e of the proximal-end-side component 22, thus regulating backward movement of the spiral tube 23.

Thus, the spiral tube 23 is configured so that the front end portion 23da and the rear end portion 23db are latched by the rear face portion 21dd of the protrusion portion 21d on the front end side and by the front face portion 22e of proximal-end-side component 22 on the rear end side, respectively. This permits the spiral tube 23 to constantly maintain such a state as to cover the external face side of the elastic cover tube 21.

On the other hand, the proximal-end-side component 22 of the insertion portion cover 10 is a tubular member having a larger diameter than that of the elastic cover tube 21 and is formed of resin material having high slidability, for example, polyacetal. Inside the proximal-end-side component 22, the through hole 22a is bored so that a part of the endoscope operation portion 12 of the endoscope 2 (a part of a bend preventing portion 12a) on the distal end side may be inserted.

On an inner-peripheral surface of the through hole 22a of the proximal-end-side component 22 on the rear end side, there are protruded the plurality of latching protrusion portions 22b formed so as to protrude inward. The latching protrusion portion 22b is fitted into a peripheral groove 12b formed in the bend preventing portion 12a of the operation portion 12 when the endoscope insertion portion 11 is inserted into the elastic cover tube 21 and a part of the operation portion 12 on the distal end side is positioned inside the proximal-end-side component 22. This permits the latching protrusion portion 22b to secure the endoscope 2 onto the introductory tube 20. A front face portion 22e of the proximal-end-side component 22 is fitted onto a part of the proximal-end portion 21 e of the elastic cover tube 21. This permits the elastic cover tube 21 to be formed integrally with the proximal-end-side component 22.

On the other hand, as shown in FIG. 5, the rotation mechanism portion 42 has a rotation section body 43 as a housing, a motor 44, a torque transmission member 45 and an introductory tube (guide tube) retainer 46. The motor 44 produces a driving force for rotating the spiral tube 23 around the longitudinal axis (hereinafter referred to as “around an axis”) of the introductory tube (guide tube) in a predetermined direction. The motor 44 is secured onto, for example, a side wall of a rotation section body 43.

On a motor shaft 44a of the motor 44, the torque transmission member 45 is integrally fixed. The torque transmission member 45 is formed of flexible resin material. The introductory tube (guide tube) retainer 46 is disposed so as to face the torque transmission member 45 fixed on the motor shaft 44a. The introductory tube (guide tube) retainer 46 is secured onto, for example, the bottom of the rotation section body 43. A flat portion facing the torque transmission member 45 of the introductory tube (guide tube) retainer 46 is formed with a semicircular recessed portion (not shown) substantially coinciding with an external shape of the spiral tube 23 or the proximal-end-side component 22.

In the rotation mechanism portion 42, as shown in FIG. 5, the spiral tube 23 constituting the introductory tube 20 is disposed in a sandwiching manner between the torque transmission member 45 and a recessed portion in the introductory tube (guide tube) retainer 46. Accordingly, the spiral tube 23 of the introductory tube 20 in such a state as shown in FIG. 3, that is, in such a state that the endoscope insertion portion 11 is inserted into the elastic cover tube 21 and the latching protrusion portion 22b is fitted into the peripheral groove 12b, is disposed between the torque transmission member 45 and the introductory tube (guide tube) retainer 46 as shown in FIG. 5. When the motor 44 is driven in this state, the torque transmission member 45 fixed on the motor shaft 44a rotates and rotation driving force is transmitted to the spiral tube 23 through the torque transmission member 45.

The spiral tube 23 to which torque is transmitted is configured so as to rotate around an axis of the elastic cover tube 21 in a clearance 23c formed between an inner-peripheral surface of the spiral-shaped portion 23b and the elastic cover tube 21. The rotation of the spiral tube 23 produces such a propulsive force that a male screw moves with respect to a female screw in a contact portion between the spiral-shaped portion 23b and the body-cavity inner wall. This permits the spiral tube 23 to attempt to move in an axial direction of the introductory tube 20 while rotating.

At this time, the position of one end (front end portion 23da) of the spiral tube 23 is regulated at an abutment position against the protrusion portion 21d on the elastic cover tube 21, while the other end (rear end portion 23db) thereof is regulated at an abutment position against the front face portion 22e of the proximal-end-side component 22. This integrates the spiral tube 23 with the elastic cover tube 21. Accordingly, the elastic cover tube 21 is configured so as to further moves in the same direction as the spiral tube 23 as the spiral tube 23 further moves.

In addition, the elastic cover tube 21 and the endoscope 2 are integrated by fitting the latching protrusion portion 22b to the peripheral groove 12b. Accordingly, the endoscope 2 is configured so as to move in the same direction as a movement direction of the introductory tube 20 constituted of the spiral tube 23 and the elastic cover tube 21 and advance toward a deep region of a body-cavity inner tube line.

The spiral tube 23 needs enlargement of its outside diameter to bring it into contact with, for example, the inner wall of a large intestine. The enlargement of the outside diameter will increase a winding length (a winding length of a metal strand 23a) per one turn of the spiral-shaped portion 23b, so that a contact area per one turn between the spiral-shaped portion 23b and the intestine wall increases, thus increasing frictional force per one turn between the spiral-shaped portion 23b and the intestine wall.

Accordingly, the spiral tube 23 needs an increase in torque per one turn for the increased frictional force, which causes the difficulty in acquiring a propulsive force from the spiral-shaped portion 23b for a constant torque, and thus the spiral tube may not advance. The present embodiment is configured so as to provide frictional force reduction means for reducing a frictional force of the spiral-shaped portion 23b of the spiral tube 23 against the body-intestine inner wall.

Referring now to FIG. 6, the configurational property of the spiral-shaped portion 23b will be described below.

As shown in FIG. 6, the spiral-shaped portion 23b is defined by a spiral pitch (hereinafter referred to as a “pitch”) P, a pitch angle PA and a strand diameter D. The pitch P refers to a distance obtained by connecting the centers of spirals adjacent to each other, the pitch angle PA refers to a spirally winding angle (tilt angle) to the longitudinal center axis, and the strand diameter D is a strand diameter of a metal strand constituting a spiral.

Next, the configuration of the spiral tube 23 according to the present embodiment will be described blow.

As shown in FIGS. 7 and 8, the spiral tube 23 according to the present embodiment has a friction reduction spiral portion 51 as frictional force reduction means at a predetermined position of the outer periphery of the spiral-shaped portion 23b, for example, on the proximal-end side.

The friction reduction spiral portion 51 is bonded with a friction reduction strand 51 a thinner and more flexible than metal strand 23a constituting the spiral-shaped portion 23b, such as silicone, polyurethane or stainless, spirally wound around the outer periphery of the spiral-shaped portion 23b.

A strand diameter D51 of the friction reduction strand 51a of the friction reduction spiral portion 51 is formed so as to be, for example, substantially ½ times as large as a strand diameter D23 of the metal strand 23a of the spiral-shaped portion 23b. In the case of an outside diameter of the spiral tube 23 of 10 mm, the strand diameter D51 of the friction reduction strand 51a is formed so as to be, for example, 1-2 mm. In this case, the strand diameter D23 of the metal strand 23a of the spiral tube 23 is, for example, 2-4 mm.

A pitch angle PA51 of the friction reduction spiral portion 51 is, for example, 120°, while a pitch angle PA23 of the spiral-shaped portion 23b is, for example, 105°. A pitch P51 of the friction reduction spiral portion 51 is set so as to be, for example, almost 3 times as large as a pitch P23 of the spiral-shaped portion 23b, so that a traveling distance per one turn, that is, a lead L51 becomes almost 3 times as large as a lead L23 of the spiral-shaped portion 23b. Accordingly, in this case, the spiral tube 23 causes a propulsive amount per one turn to be almost 3 times as large as in a case where only the spiral-shaped portion 23b is installed.

This permits the spiral tube 23 to have such a state that a contact area per one turn of the friction reduction spiral portion 51, (D51/D23)×(L51/L23)=D51L51I/D23L23 becomes, for example, around (½)×(⅓)=around ⅙ times as large as a contact area per one turn of the spiral-shaped portion 23b. Accordingly, the spiral tube 23 decreases a frictional force compared with the case of the spiral-shaped portion 23b because a contact area between the friction reduction spiral portion 51 and a body-cavity inner wall becomes smaller than a contact area between the spiral-shaped portion 23b and the body-cavity inner wall.

The friction reduction spiral portion 51 permits a close contact of the friction reduction strand 51a to be changed by decreasing or increasing the pitch P51 and the spiral angle PA51 to be variously set regardless of the spiral shape. Preferably, the friction reduction spiral portion 51 is formed by winding the friction reduction strand 5 la in the same left-handed spiral manner as for the metal strand 23a of the spiral-shaped portion 23b.

The operation of an endoscope system 1 configured in the above way will be described below.

First, a medical staff (hereinafter referred to as “staff”) prepares the endoscope 2 and the introductory tube 20 constituting the endoscope insertion assisting tool 3, moves the arm portion 41 of the rotation device 40 constituting the endoscope insertion assisting tool 3 and locates the rotation mechanism portion 42 at a desired position.

Next, the staff locates a desired portion of the spiral tube 23 constituting the introductory tube 20 between the introductory tube (guide tube) retainer 46 and the torque transmission member 45 constituting the rotation mechanism portion 42. This allows the proximal end portion side of the introductory tube 20 to be retained by the rotation mechanism portion 42. At this time, the staff locates the distal end portion side of the introductory tube 20, for example, on a bed 7.

Next, the staff inserts the endoscope insertion portion 11 into the introductory tube 20 from an opening in the proximal-end-side component 22 constituting the introductory tube 20. This permits the endoscope insertion portion 11 to be covered with the introductory tube 20 to complete preparation for insertion of the endoscope 2, for example, into a large intestine. The staff prepares the light source 4, the video processor 5 and the monitor 6 as peripheral apparatuses together with the endoscope 2, the introductory tube 20 and the rotation device 40.

Next, a step of inserting the endoscope 2 covered by the introductory tube 20 into a large intestine will be described below. First, an operator (not shown) holds the distal end of the introductory tube 20 and inserts the distal end of the introductory tube 20 into the large intestine of a patient 8 lying on a bed 7 from the anus. And the spiral-shaped portion 23b formed on an outer surface of the spiral tube 23 provided in the introductory tube 20 comes into contact with the intestine wall. At this time, a contact state between the spiral-shaped portion 23b and the intestine wall has such a relationship as seen in an external thread and an internal thread. On a screen of the monitor 6, an endoscope image picked up by an image pickup device of the endoscope 2 through the observation window 18 is displayed.

The operator rotates the motor 44 of the rotation mechanism portion 42 by a predetermined operation under such a condition that the spiral-shaped portion 23b comes into contact with the intestine wall. And the torque transmission member 45 rotates through a motor shaft 44a of the motor 44. This permits a rotational driving force to be transmitted to the spiral tube 23 disposed between the torque transmission member 45 and the introductory tube (guide tube) retainer 46. Accordingly, as indicated by an arrow R in FIG. 9, the spiral tube 23 starts rotation around the axis.

At this time, a contact portion between the spiral-shaped portion 23b of the rotating spiral tube 23 and the intestine wall has such a relationship that an external thread moves to an internal thread, that is, generates a propulsive force for advancing the spiral tube 23. As described above, the position of one end (front end portion 23da) of the spiral tube 23 is regulated at an abutment position against the protrusion portion 21 d of the elastic cover tube 21, while the position of the other end (rear end portion 23db) is regulated at an abutment position against the front face portion 22e of the proximal-end-side component 22, so that the spiral tube 23 is integrated with the elastic cover tube 21.

Accordingly, the spiral tube 23 is made to abut against the rear face portion 21dd of the protrusion portion 21d of the elastic cover tube 21 and advances while pressing it, without dropping off the elastic cover tube 21. This permits the introductory tube 20 constituted of an elastic cover tube 21 and the spiral tube 23 to be advanced toward the depth region of the large intestine by the propulsive force.

In the proximal-end-side component 22 of the introductory tube 20, fitting the peripheral groove 12b onto the latching protrusion portion 22b integrates the endoscope 2. Accordingly, as the introductory tube 20 moves, the endoscope 2 as well moves in the same direction and introduced toward the deep region of the body cavity of a subject. The spiral tube 23 has the friction reduction spiral portion 51 as frictional force reduction means at a predetermined position of the outer periphery of the spiral-shaped portion 23b as described above. Accordingly, as insertion of the introductory tube into the body cavity advances, the friction reduction spiral portion 51 comes into contact with the body-cavity inner wall in place of the spiral-shaped portion 23b and is rotated by the torque transmitted from the spiral-shaped portion 23b. The spiral-shaped portion 23b has excellent snapping capability, and high resistance to bending and good rotation transmission to the friction reduction spiral portion 51.

The friction reduction spiral portion 51, in which a propulsive force for advancing the spiral tube 23 is produced like the spiral-shaped portion 23b and the propulsive force is transmitted to the introductory tube 20 as described above through the spiral-shaped portion 23b, is introduced toward the depth region of the body cavity of the subject together with the endoscope 2.

That is to say, the introductory tube 20 inserted from an anus 71 in which the endoscope insertion portion 11 is inserted is advanced toward an S-shaped colon portion 73 from a rectum 72 by the propulsive force and operator's manual operation and bending operation. The introductory tube 20 passes through the S-shaped colon portion 73, a bending portion as a boundary between the S-shaped colon portion 73 and colon descendens 74 with low movability, a splenic flexture portion 76 as a boundary between colon descendens 74 and a transverse colon portion 75 with high movability and a liver bending portion 77 as a boundary between the transverse colon portion 75 and colon ascendens 78. Then, the introductory tube 20 reaches around a caecum portion 79 as a target portion.

At this time, the friction reduction spiral portion 51 is set so that a contact area with the body-cavity inner wall per one turn is substantially ⅙ times as large as the spiral-shaped portion 23b. Accordingly, the spiral tube 23 decreases a frictional force working against the intestine wall compared with the case of the spiral-shaped portion 23b because a contact area between the friction reduction spiral portion 51 and the body-cavity inner wall becomes smaller than a contact area between the spiral-shaped portion 23b and the body-cavity inner wall.

This permits the spiral tube 23 to prevent an increase in a frictional force working against the intestine wall, thus attaining a satisfactory propulsive force. Accordingly, the introductory tube 20 provides a significant propulsion function while being inserted into the body cavity, thus facilitating insertion of the endoscope insertion portion 11 into the body cavity.

If an operator performs manual operation of pushing forward the introductory tube 20 under such a condition, the introductory tube 20 with the endoscope insertion portion 11 inserted therein is introduced toward the depth region of the body cavity with a little effort. On the introductory tube 20, for example, filth or the like may adhere to the observation window member 24. In this case, the operator depresses a push-button switch 28 for air/water supply twice. And the introductory tube 20 jets, for example, water as indicated by an arrow from the opening in the air/water supply nozzle 25 through the through hole 21b by starting the air/water supply apparatus 27 to supply water. This permits the introductory tube 20 to wash away filth or the like adhering to the observation window member 24.

Moreover, the operator depresses the push-button switch 28 for air/water supply once. And the introductory tube 20 jets, for example, air as indicated by an arrow from the opening in the air/water supply nozzle 25 through the through hole 21b by starting the air/water supply apparatus 27 to supply air. This permits the introductory tube 20 to remove water droplets adhering to a surface of the observation window member 24. Furthermore, the operator depresses the push-button switch 29 for suction. And the introductory tube 20 sucks body fluid or the like from the opening of the through hole 21 c by starting the suction apparatus.

Then, the operator, after determining that a distal end portion of the introductory tube 20 reaches around the caecum portion 79 from an endoscope image displayed on a screen of the monitor 6, gives an instruction, for example, to a staff to stop driving of the motor 44. At this time, to perform inspection of the internal large intestine, the operator shifts to pulling-back of the endoscope insertion portion 11 for the inspection. After completion of the inspection, the operator removes the endoscope insertion portion 11 from the introductory tube 20, scraps the introductory tube 20 and inserts the endoscope insertion portion 11 into a new introductory tube 20 before use. This permits the next inspection without need of cleaning and sterilizing the endoscope 2.

As the result, because the friction reduction spiral portion 51 is provided at a predetermined position of the outer periphery of the spiral-shaped portion 23b as frictional force reduction means, the friction reduction spiral portion 51 comes into contact with the body-cavity inner wall in place of the spiral-shaped portion 23b, so that a frictional force working against the body-cavity inner wall can be reduced and a satisfactory propulsion function can be exhibited, thus facilitating insertion of the endoscope insertion portion 11 into the body cavity.

Insertion of the endoscope insertion portion into the introductory tube 20 enables to completely prevent the endoscope insertion portion 11 from coming into direct contact with the body cavity during the inspection. Accordingly, reuse in a combination of the endoscope removed from the introductory tube with a new introductory tube without cleaning and sterilizing after completion of the inspection can relieve medical staff of troublesome cleaning and sterilizing of the endoscope and the introductory tube at every inspection completion.

In the present embodiment, a large intestine is taken as a tubular body cavity in which the endoscope insertion portion covered with the introductory tube 20 is inserted, but a tubular body cavity in which the endoscope insertion portion 11 is inserted is not limited to the large intestine, which may be any of tubular body cavities from oral cavity to esophagus, stomach and small intestine.

The rotational direction of the introductory tube 20 in the present embodiment may be only one way (advance direction) or clockwise/counterclockwise rotation may be performed in a fixed cycle or by arbitrary switching. Combinations of clockwise/counterclockwise rotations permits the introductory tube 20 to repeat forward and rearward movement in the body cavity. Even if the distal end of the introductory tube 20 is caught in a small recessed portion or the like in the intestine wall during forward movement, the catch can be relieved during reward movement. During the second forward movement, the positions of the intestine and the introductory tube 20 are finely dislocated from each other, which permits smooth advance without causing recurrence of the catch.

In the present embodiment, the present invention is applied to a configuration of a disposable sheath as the introductory tube 20, but the present invention is not limited thereto. Naturally, the present invention may be applied to a full-disposable type formed integrally with an endoscope insertion portion as an introductory tube and an over-tube for appropriate endoscope as a tubular-shaped tube formed so as to be harder than a flexible tube portion of an endoscope (hereinafter referred to as “over-tube”). In short, it is sufficient that formation of the friction reduction spiral portion 51 can reduce a frictional force between a large-diameter spiral tube 23 and a body-cavity inner wall to provide a substantial propulsion function.

### Second Embodiment

Referring next to drawings, a second embodiment of the present invention will be described below.

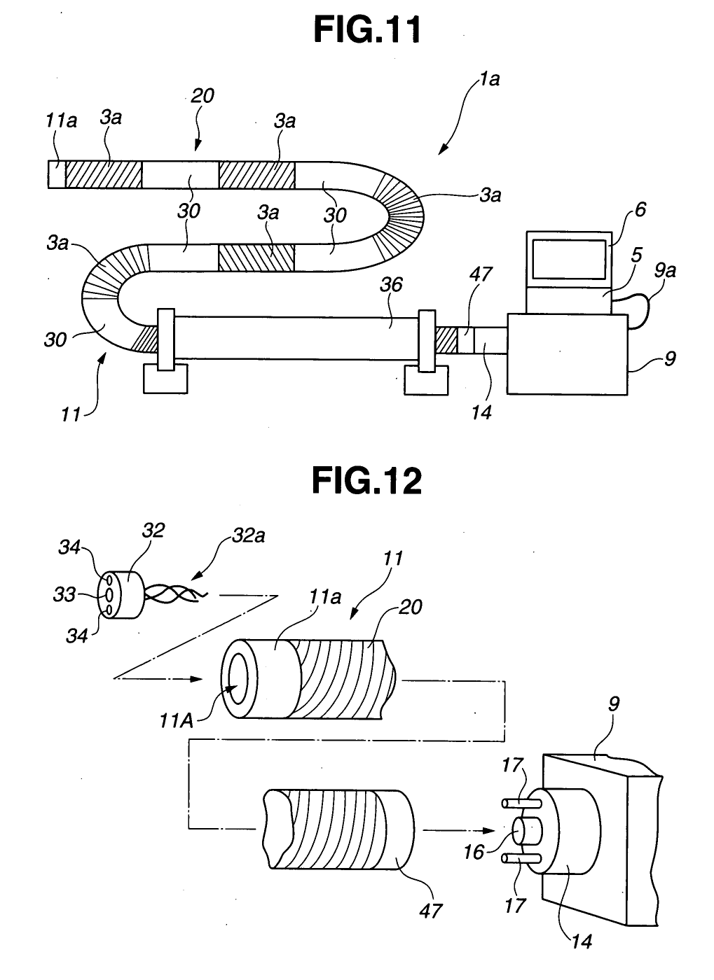

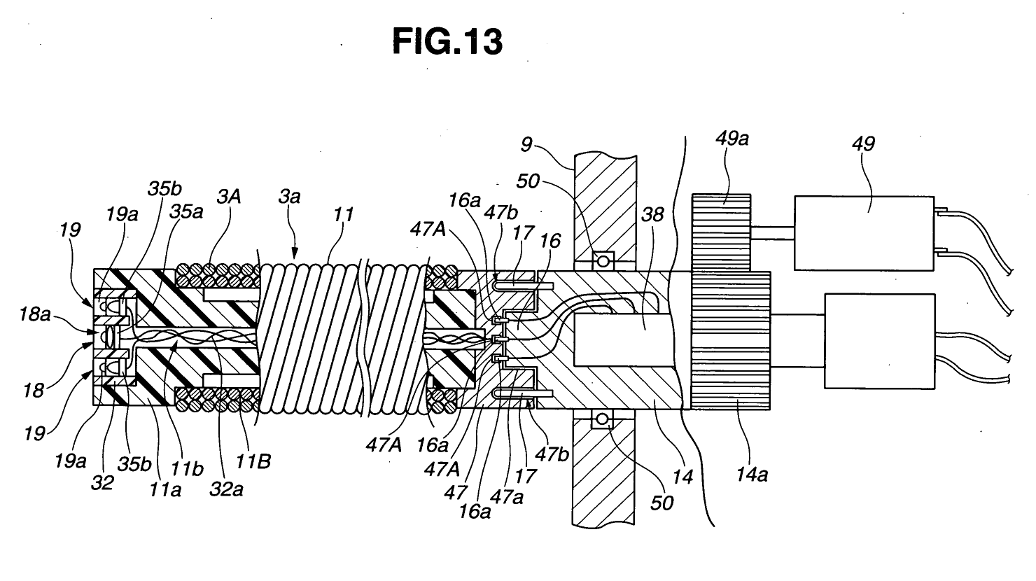

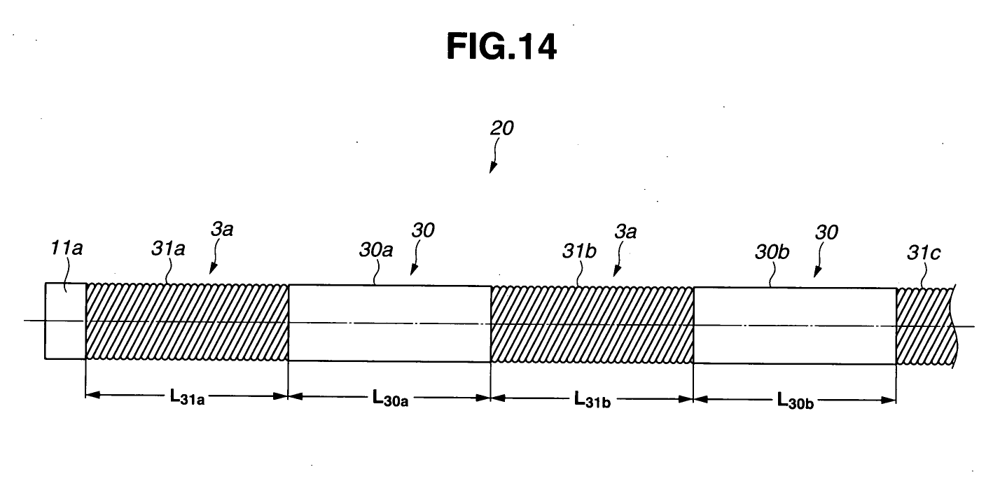

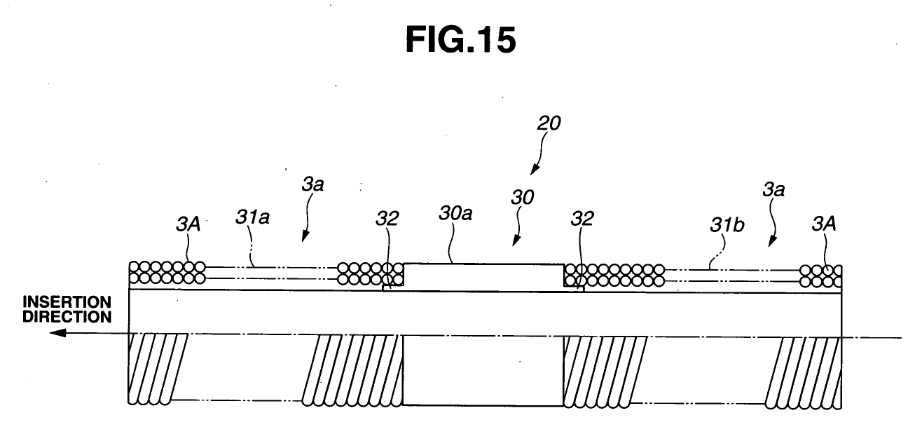

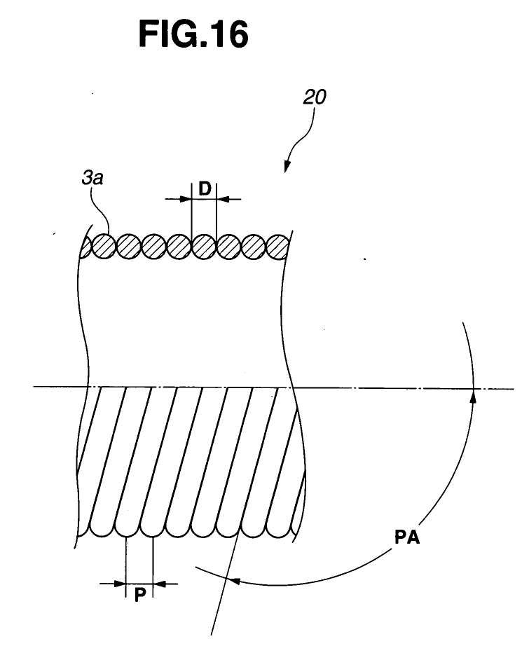

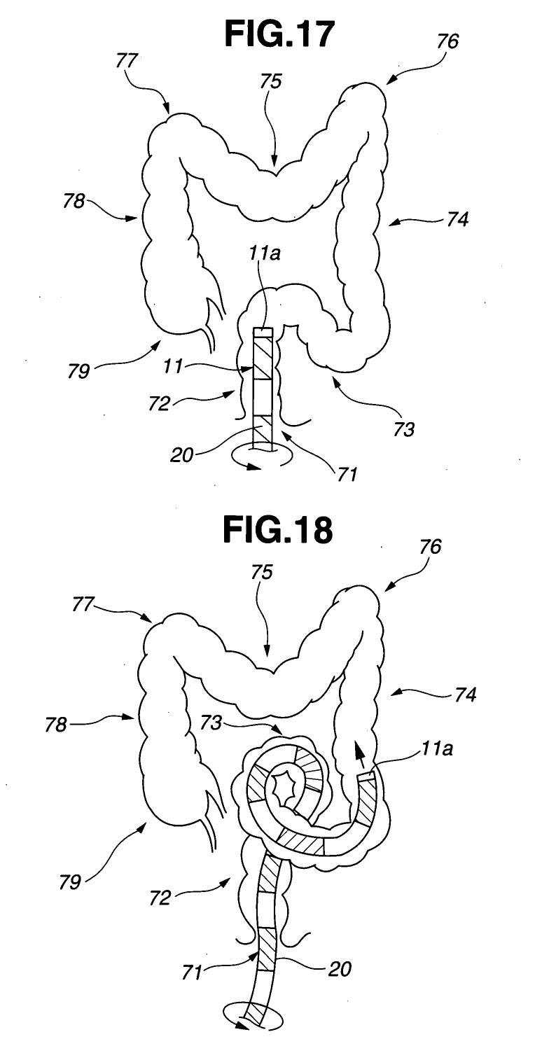

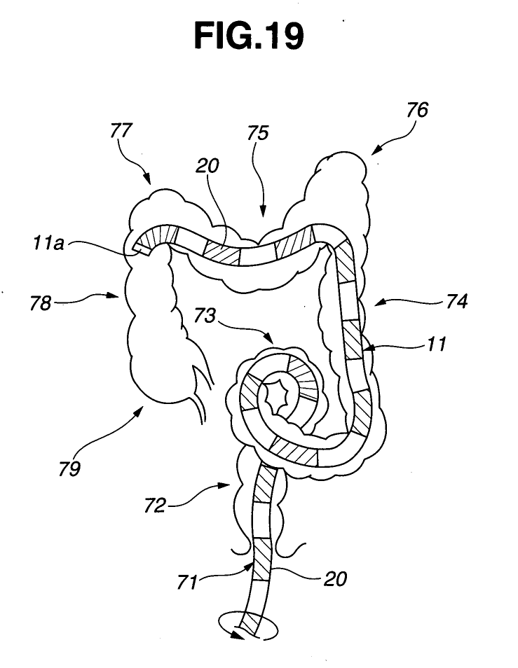

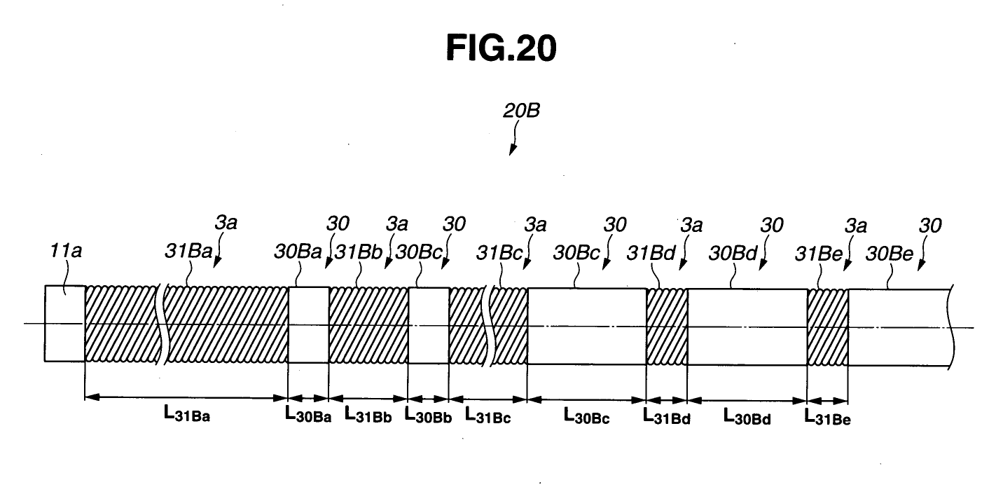

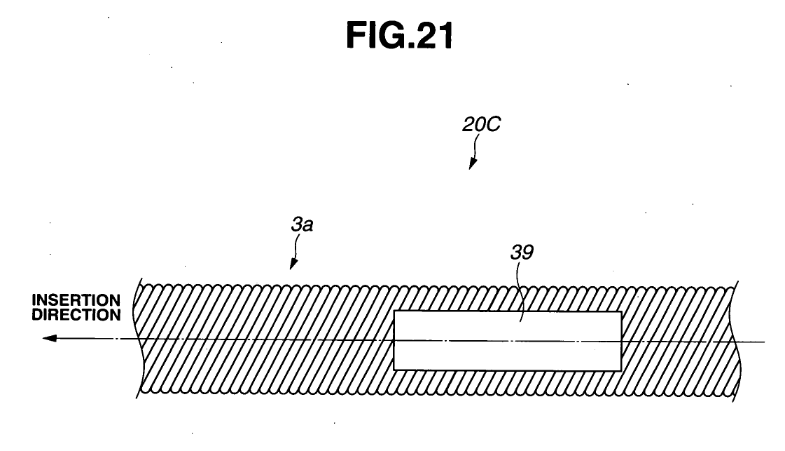

FIGS. 11 to 20 are views of an endoscope insertion portion and an endoscope system according to the present invention, FIG. 11 is a view showing an overall configuration of an endoscope system in a second embodiment, FIG. 12 is a descriptive view showing connection of an endoscope insertion portion with an endoscope rotation device (referred to as a “rotation device”) in FIG. 11, FIG. 13 is a partially sectional view with the endoscope insertion portion in FIG. 12 cut in a longitudinal direction, FIG. 14 is an external view of the introductory tube (guide tube) in FIG. 13; FIG. 15 is a descriptive view showing the introductory tube (guide tube) in FIG. 14; FIG. 16 is a descriptive view of an endoscope insertion portion inserted into a large intestine, FIG. 17 is a descriptive view showing such a state that an endoscope insertion portion is inserted while an S-shaped colon portion is being drawn into an α-loop shape, FIG. 18 is a descriptive view of an endoscope insertion portion inserted into a deep region of a large intestine, FIG. 19 is an external view of the introductory tube (guide tube) showing a deformed example of FIG. 14, FIG. 20 is an external view of an introductory tube (guide tube) configured by attaching covering tape at a predetermined position as covering means, and FIG. 21 is a descriptive view showing the configurational property of a spiral-shaped portion.

As shown in FIG. 11, an endoscope system 1a in the present embodiment is mainly constituted of: an endoscope insertion portion 11; a rotation device 9 for rotating the endoscope insertion portion 11 around the longitudinal axis in a predetermined direction, a protection tube 36 retaining rotation of the endoscope insertion portion 11; a video processor 5 connected to the rotation device 9 by a cable 9a and described in the first embodiment; and a monitor 6 displaying an image picked up by the endoscope insertion portion 11 and described in the first embodiment.

The video processor 5 has a signal processing circuit. The video processor 5 transmits a driving signal for driving an image pickup device 16 described later built in the endoscope insertion portion 11, converts an electric signal photoelectrically-converted and transferred by an image pickup device to a video signal and outputs the video signal to the monitor 6. The monitor 6, after receiving the video signal outputted from the video processor 5, displays an endoscope image on its screen.

The endoscope insertion portion 11 has an introductory tube (guide tube) 20 which is a propulsive force generation section as an insertion-portion guide portion between an endoscope distal end portion (hereinafter referred to as a “distal end”) 1 la and a connector portion 47 and is loosely inserted into the protection tube 36 for preventing it from coming into contact with a floor in an operation room. This permits the endoscope insertion portion 11 to be prevented from coming into direct contact with a floor or the like. The connector portion 47 of the endoscope insertion portion 11 is connected with an insertion portion retainer 14 as a substantially cylindrical body protruding from one side surface of the rotation device 9.

As shown in FIG. 12, the distal end 11a of the endoscope insertion portion 11 has a camera unit storage portion 11A as a substantially cylindrical hole portion. The camera unit storage portion 11A stores and secures a camera unit 32. The camera unit 32 includes an observation optical system and an illumination optical system as an image pickup unit.

The insertion portion retainer 14 of the rotation device 9 has a substantially cylindrical protrusion portion 16 protruding from the distal end face and a plurality of pins 17 (two, in the figure). The insertion portion retainer 14 is configured so as to be connected with the endoscope insertion portion 11 by fitting the pin 17 and the protrusion portion 16 to the connector portion 47 of the endoscope insertion portion 11. The camera unit 32 has an observation window 18 substantially in the center of the distal end face, illumination windows 19 near the observation window 18, and an electric cable 32a inserted into the endoscope insertion portion 11 from its proximal end side.

Referring next to FIG. 13, the endoscope insertion portion 11 and the rotation device 9 will be described in detail later.

As shown in FIG. 13, the camera unit 32 secured onto the distal end 11a has an observation optical system 18a disposed behind the observation window 18; an image pickup device (hereinafter referred to as “CCD”) 35a disposed behind the observation optical system 18a; two illumination optical systems 19a disposed respectively rearward of the two illumination windows 19; and two light emitting diodes (hereinafter referred to as “LED”) 35b disposed respectively rearward of the illumination optical systems 19a.

An image signal cable connected to CCD35 and an electric cable 32a connected to LED35b extend from the proximal end side of the camera unit 32. Preferably, the image signal cable and the LED power cable have almost the same voltage, which can prevent various types of damage due to the respective cables in proximity to each other, such as damage to CCD35 and LED35b due to electromagnetic induction.

An insertion portion body 11B is formed with a through hole 11b with an electric cable 32a extending from the camera unit 32 inserted therethrough. The insertion portion body 11B, having the distal end 11a of single collar type, is a substantially cylindrical body having flexibility. The insertion portion body 11B has a proximal end secured to the connector portion 47 and the introductory tube (guide tube) 20 sheathed.

The introductory tube (guide tube) 20 is a tube formed so as to have a predetermined flexibility by spirally winding two turns of metal strand 3A having a predetermined diameter, for example, made of stainless, around the outer periphery of the insertion portion body 11B between the distal end 11a of the endoscope insertion portion 11 and the connector portion 47.

The introductory tube (guide tube) 20 may be formed by spirally winding metal strand 3A in a plurality of rows, for example, 4 rows. The metal strand 3A wound in a spiral manner can improve close contact between metal strands and variously set a variety of spiral angles. Accordingly, on an outer surface of the introductory tube (guide tube) 20, there is provided a spiral-shaped portion 3a formed by a surface of the metal strand 3A. Preferably, the metal strand 3A is formed by being wound in a left-handed spiral manner toward the proximal end from the distal end. In other words, it is preferable to spirally wind the metal strand 3A in the same direction as a left-handed thread groove and specifically, to rotate the insertion portion retainer 14 of the rotation device 9 counterclockwise around the longitudinal axis of the endoscope insertion portion 11 in inserting the metal strand into the body cavity, especially large intestine, thus attaining high close contact with the inner wall of the large intestine and high insertability of the endoscope insertion portion 11 into the large intestine.

The connector portion 47 has a fitting hole 47a as a substantially columnar hole substantially in the center of a proximal-end surface and two pin holes 47b in the periphery of the fitting hole 47a. Accordingly, in the connector portion 47, the protrusion portion 16 of the insertion portion retainer 14 is inserted into the fitting hole 47a and the two pins 17 of the insertion portion retainer 14 are inserted into the two pin holes 47b respectively for positive connection with the insertion portion retainer 14.

The fitting hole 47a has three contact terminals 47A on the end face and the contact terminals 47A and a plurality of electric cables 32a are connected with each other. In connecting the connector portion 47 with the insertion portion retainer 14, the three contact terminals 47A of the connector portion 47 come into contact with three contact pins 16a of the protrusion portion 16 of the insertion portion retainer 14 respectively, thus electrically connecting CCD35 and LED35b with the rotation device 9.

The insertion portion retainer 14 has a current collector 38 (hereinafter referred to as a “slip ring”) having the same center axis as a rotational axis and is retained so as to rotate around the longitudinal axis together with a side plate of the rotation device 9, for example, by a bearing 50. Moreover, the insertion portion retainer 14 is formed with, for example, a spur-gear-shaped gear groove 14a on the outer periphery of a proximal-end portion.

The insertion portion retainer 14 engages with a spur gear 49a in which the gear groove 14a at the proximal-end portion is formed at the distal end of a motor shaft of a motor 49 and is configured so as to rotate around a predetermined longitudinal axis direction or, in the present embodiment, rotate counterclockwise toward the distal end from the proximal end by the motor 49.

Hence, in performing insertion into the body cavity, especially a large intestine, the insertion portion retainer 14 is rotated by the rotation device 9, so that the introductory tube (guide tube) 20 provides such a propulsive force that an external thread works on an internal thread by the spiral-shaped portion 3a rotating in a close contact with the inner wall of the large intestine. In the introductory tube (guide tube) 20, the spiral-shaped portion 3a is formed on an outer-peripheral surface of the endoscope insertion portion 11 over the whole periphery from the rear end side of the distal end 11a to the connector portion 47.

The endoscope insertion portion 11 inserted into the body cavity may unavoidably obtain the above-described propulsive force due to the spiral-shaped portion 3a coming into a close contact with the inner wall of the large intestine over the predetermined portion inserted into the body cavity from the rear end side of the distal end 11a. And, as the endoscope insertion portion 11 brings its insertion nearer to the depth region of the body cavity, a length of the spiral-shaped portion 3a obtaining a propulsive force in a close contact with the inner wall of the large intestine becomes larger, so that the spiral-shaped portion 3a having a larger length increases a propulsive force for further insertion into the body cavity. Accordingly, the introductory tube (guide tube) 20 obtains a higher propulsive force than is necessary for an operator to a degree that the intestine is expanded to excessively change a running condition of the intestine, which causes low insertability into the body cavity.

The spiral-shaped portion 3a is a coiled body closely wound in a plurality of rows as described above and has difficulty in processing (manufacturing). Especially, as its length is larger, the degree of the difficulty becomes higher, which may cause low processing capability. The present embodiment is configured so as to provide propulsive force reduction means for reducing a propulsive force of the introductory tube (guide tube) 20 for the introductory tube (guide tube) 20.

That is, as shown in FIGS. 14 and 15, the introductory tube (guide tube) 20 is configured so that the spiral-shaped portions 3a are linked with connection elements 30 (30a, 30b, . . .) at every predetermined interval as propulsive force reduction means. Specifically, the introductory tube (guide tube) 20 is constituted of: a spiral-shaped portion 31a, a connection element 30a, a spiral-shaped portion 31b, a connection element 30b, a spiral-shaped portion 31c and so on.

The connection elements 30a, 30b, . . . are respectively bonded with the spiral-shaped portions 3a (31a, 31b, 31c, . . .) at a stepped portion 37 on both sides of each of them and are configured so as to rotate together with the spiral-shaped portions 3a (31a, 31b, 31c, . . .).

The connection element 30 is formed of a flexible member, for example, a polyurethane tube or fluororesin such as PTFE (tetrafluoroetylene resin). The surface of each of the connection elements 30 (30a, 30b, . . .) is smooth, frictional coefficient μ of which is, for example, 0.015 to 0.020.

The frictional coefficient μ is a ratio of a frictional force working on a contact surface between two objects in parallel to a vertical drag (pressure) working on the surface at right angles.

The structural property of the spiral-shaped portion 3a is as shown in FIG. 16. As shown in FIG. 16, the spiral-shaped portion 3a is defined by a spiral pitch (hereinafter referred to as a pitch) P, a pitch angle PA and a strand diameter D. The pitch P refers to a distance obtained by connecting the centers of spirals adjacent to each other, the pitch angle refers to a spirally winding angle (tilt angle) to the longitudinal center axis, the strand diameter is a diameter of a metal strand constituting a spiral, and a depth of the spiral groove is an angle of a groove formed between adjacent spirals.

In the present embodiment, the pitches P, pitch angles PA and strand diameters D of the spiral-shaped portions 31a, 31b, 31c, . . . are all formed so as to be the same.

In the introductory tube (guide tube) 20, lengths L31a, L31b, L31c, . . . of the spiral-shaped portions 31a, 31b, 31c, . . . and lengths L30a, L30b, . . . of the connection elements 30a, 30b are formed so as to have the same length respectively. That is, the introductory tube (guide tube) 20 is configured so that the spiral-shaped portion 3a and the connection element 30 are alternately provided in a linked manner and their component ratio is 1:1. Accordingly, the introductory tube (guide tube) 20 in the present embodiment provides a frictional force of approx. ½ and an obtained propulsive force of approx. ½ as compared to a case where the spiral-shaped portion 3a is formed on an outer-peripheral surface of the endoscope insertion portion 11 over the whole periphery from the rear end of the distal end 11a to the connector portion 47.

The introductory tube (guide tube) 20 in the present embodiment can attain reduction in the configuration of the spiral-shaped portion 3a to a half as well as high processing capability and weight reduction as compared to a case where the spiral-shaped portion 3a is formed on an outer-peripheral surface of the endoscope insertion portion 11 over the whole periphery from the rear end of the distal end 11a to the connector portion 47.

The operation of an endoscope system 1a according to the present embodiment configured in the above way will be described below. The step of preparing for inserting the endoscope insertion portion 11 into the large intestine will be described below.

In inserting the endoscope insertion portion 11 into a large intestine, for example, to a caecum portion, first, a doctor or a nurse (hereinafter referred to as an “operator”) inserts the endoscope insertion portion 11 into a protection tube 36. Then, the operator connects the connector portion 47 of the endoscope insertion portion 11 projecting from the protection tube 36 with the insertion portion retainer 14 of the rotation device 9. At this time, the operator inserts the two pins 17 of the insertion portion retainer 14 into the two pin holes 47b of the connector portion 47 respectively and fits the protrusion portion 16 of the insertion portion retainer 14 into the fitting hole 47a of the connector portion 47. Here, the preparation for inserting the endoscope insertion portion 11 into the large intestine of a patient is completed. Moreover, the operator prepares for the video processor 5 and the monitor 6 in addition to the endoscope insertion portion 11.

Referring now to FIGS. 17 and 18, the step of inserting the endoscope insertion portion 11 into the large intestine will be described below.

First, the operator grasps the distal end portion of the endoscope insertion portion 11 and inserts the distal end 1 a of the endoscope insertion portion 11 into the large intestine from the anus 71 (see FIG. 17) of the patient lying on a bed or the like. And the spiral-shaped portions 3a (31a, 31b, 31c, . . .) formed on an outer surface of the endoscope insertion portion 11 comes into contact with the intestine wall of the patient. At this time, a contact condition between the spiral-shaped portion 3a formed on the endoscope insertion portion 11 and the intestine wall has the same relationship as that between an external thread and an internal thread.

Under such a contact condition, the operator sets the motor 49 of the rotation device 9 in such a driving condition as to be rotated around the axis of the endoscope insertion portion 11 counterclockwise. And the endoscope insertion portion 11 rotates around the axis counterclockwise toward the insertion direction, and the connector portion 47 of the endoscope insertion portion 11 mounted on the insertion portion retainer 14 rotates around the axis counterclockwise toward the insertion direction. The rotation is alternately transmitted to the spiral-shaped portion 3a and the connection element 30 from the proximal-end portion of the endoscope insertion portion 11 and reaches the distal end 11a, and the endoscope insertion portion 11 rotates around the axis counterclockwise.

This generates a propulsive force for advancing the endoscope insertion portion 11 as if an external thread moved with respect to an internal thread, at a contact portion between the spiral-shaped portion 3a of the rotated endoscope insertion portion 11 and the intestine wall. Then the endoscope insertion portion 11 advances to the depth region of the large intestine by the propulsive force.

At this time, the operator may manually operate the endoscope insertion portion 11 with hand so as to advance it. And, as shown in FIG. 17, the endoscope insertion portion 11 inserted from the anus 71 of the patient advances to the S-shaped colon portion 73 from the rectum 72 by the propulsive force and operator's manual operation. Then, the endoscope insertion portion 11 reaches the S-shaped colon portion 73.

The endoscope insertion portion 11, when passing through the S-shaped colon portion 73, for example, advances along the intestine wall while forming an a loop shape at the S-shaped colon portion 73 as shown in FIG. 18. When the spiral-shaped portion 3a is formed on an outer-peripheral surface of the endoscope insertion portion 11 over the whole periphery from the rear end side of the distal end 11a to the connector portion 47, all outer periphery portions in contact with the intestine wall portion in a loop shape are the spiral-shaped portion 3a. Accordingly, all the portions in contact contribute on the propulsive force to enlarge the loop, which may inhibit propulsion of the distal end.

However, in the present embodiment, the spiral-shaped portion 3a and the connection element 30 are alternately provided consecutively as described above, therefore the spiral-shaped portion 3a is configured to have a half volume as compared to a case where the spiral-shaped portion 3a is formed over the whole periphery of the endoscope insertion portion 11, thus generating no excessive propulsive force. Accordingly, the introductory tube (guide tube) 20 will not expand more than is necessary and impair insertability, thus improving insertability of the endoscope insertion portion 11 into the body cavity without giving a pain to the patient.

The endoscope insertion portion 11 passes through the S-shaped colon portion 73, advances smoothly along walls of a bending portion as a boundary between the S-shaped colon portion 73 and colon descendens 74 with low movability, a splenic flexture portion 76 as a boundary between the colon descendens 74 and a transverse colon portion 75 with high movability and a liver bending portion 77 as a boundary between the transverse colon portion 75 and colon ascendens 78, and reaches, for example, around the caecum portion 79 as a target portion without changing a condition of the large intestine as shown in FIG. 19.

During insertion of the endoscope insertion portion 11 into the large intestine of the patient, the operator inserts the endoscope insertion portion 11 to the depth region of the large intestine by a propulsive force of the endoscope insertion portion 11 and manual operation of grasping hand for advancing the endoscope insertion portion 11 while observing images of the internal large intestine displayed on a screen of the monitor 6.

At this time, the video processor 5 performs image processing so as to cause no rotating display of images displayed on the screen of the monitor 6 by rotation of the endoscope insertion portion 11. In other words, the video processor 5 performs image processing so that only a still image at a predetermined phase position synchronized with a rotation cycle of the distal end 11a of the endoscope insertion portion 11 is displayed on the screen of the monitor 6, and the video signal subjected to the image processing is transmitted to the monitor 6 and displayed on the screen of the monitor 6.

Upon determining that the introductory tube (guide tube) 20 has reached around the caecum portion 79 from endoscope images displayed on the monitor 6, the operator stops rotating the motor 49 of the rotation device 9. This stops advancing the introductory tube (guide tube) 20. Then, endoscope inspection is performed for around the caecum portion 79.

Hence, an endoscope system 1a in a second embodiment generates no excessive propulsive force as compared to a case where the spiral-shaped portion 3a is formed over the whole periphery of the endoscope insertion portion 11 because the spiral-shaped portion 3a is configured to a half. Accordingly, the endoscope system 1a in the first embodiment permits easy insertion of the endoscope insertion portion into a body cavity such as a large intestine and improvement of the endoscope insertion portion 11 into body cavities without giving a pain to the patient. Furthermore, the endoscope system 1a in the second embodiment provides high processing capability and weight reduction because the spiral-shaped portion 3a is configured to a half.

In the present embodiment, the present invention is applied to a full-disposable type introductory tube (guide tube) formed integrally with the endoscope insertion portion by providing an observation optical system (camera unit 32) as an insertion-portion guide portion, but is not limited thereto. The present invention may be applied to a disposable sheath as the introductory tube (guide tube) 20. Furthermore, the present invention may be applied to, what is called, an over-tube for endoscope as a tubular-shaped tube formed so as to be harder than a flexible tube portion of an endoscope as the introductory tube (guide tube) 20.

Moreover, introductory tube (guide tube) may be configured so as to reduce the number of spiral-shaped portions toward the proximal end side as propulsive force reduction means.

As shown in FIG. 20, the introductory tube (guide tube) 20B is configured so that only the spiral-shaped portions 3a are used in the range from the distal end to a predetermined distance, the number of the connection elements 30B (30Ba, 30Bb, . . .) is increased from a predetermined distance and the number of spiral-shaped portions 3a is reduced so as to be smaller than that of the connection elements 30B on the proximal end side.

More specifically, the introductory tube (guide tube) 20B is constituted of: a spiral-shaped portion 31Ba, a connector element 30Ba, a spiral-shaped portion 31Bb, a connector element 30Bb, a spiral-shaped portion 31Bc, a connector element 30Bd, a spiral-shaped portion 31Bd, a connection element 30Be, a spiral-shaped portion 31Be and so on. The connection element 30B is formed of a flexible member in the same way as for the connection element 30, for example, a polyurethane tube or fluororesin such as PTFE (tetrafluoroetylene resin). The surface of each of the connection elements 30 (30a, 30b, . . .) is smooth, frictional coefficient μ of which is, for example, 0.015 to 0.020.

A length L31Ba of the spiral-shaped portion 31Ba is, for example, a total length of a length L31a of the spiral-shaped portion 31a and a length L30a of the connection element 30a. Moreover, lengths L31Bb, L31Bc of the spiral-shaped portions 31Bb, 31Bc are, for example, almost twice as large as lengths L30Ba, L30Bb of the connection elements 30Ba, 30Bb. Furthermore, lengths L3lBd, L31Be of the spiral-shaped portions 31Bd, 31Be are, for example, substantially ⅓ times as large as lengths L30Bc, L30Bd of the connection elements 30Bc, 30Bd.

That is to say, the introductory tube (guide tube) 20B is configured so that spiral-shaped portions 3a are formed long by a predetermined distance for the distal end to obtain a predetermined propulsive force and easily advance, where a component ratio of the spiral-shaped portions 3a is higher than that of the connection elements 30 and the component ratio of the spiral-shaped portions 3a is lower than that of the connection elements 30 toward the proximal end side.

Accordingly, the introductory tube (guide tube) 20B makes an easier advance to the depth region of the body cavity, and a propulsive force obtained from the proximal end side is, for example, approx. ¼ times and lowers toward the proximal end side, as compared to the introductory tube (guide tube) 20. This permits the introductory tube (guide tube) 20B to attain the same effect as for the first embodiment and an appropriate propulsive force is obtained as it is inserted to the depth region of the body cavity because only the distal end obtains a propulsive force without the proximal end side obtaining an excessive propulsive force.

The introductory tube (guide tube) may be constituted by using covering means for covering the spiral-shaped portion as propulsive force reduction means.

As shown in FIG. 21, the introductory tube (guide tube) 20C is configured by attaching a predetermined length of covering tape 39 at a predetermined position as covering means.

The covering tape 39 is formed of a flexible member, for example, a polyurethane tube or fluororesin such as PTFE (tetrafluoroetylene resin) in the same way as for the connection element 30. The surface of the covering tape 39 is smooth, frictional coefficient μ of which is, for example, 0.015 to 0.020. The covering tape 39 may be formed by applying coating processing for high lubrication capability.

In FIG. 21, the covering tape 39 is attached onto a predetermined position of the spiral-shaped portion 3a, for example, using adhesives. However, the covering tape 39 may be, not shown, configured so as to cover a predetermined portion of the spiral-shaped portion 3a by winding in the same manner as the spiral-shaped portion 3a. This permits the introductory tube (guide tube) 20C to attain the same effect as for the first embodiment and simple configuration only by attaching the covering tape 39.

### Third Embodiment

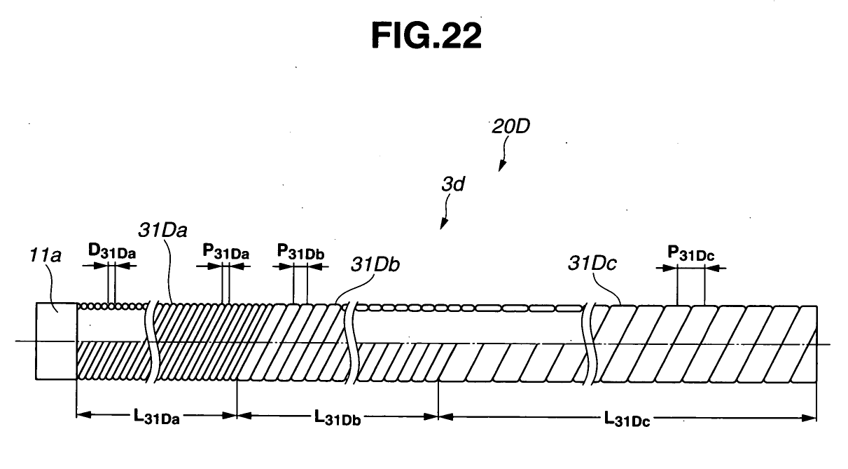

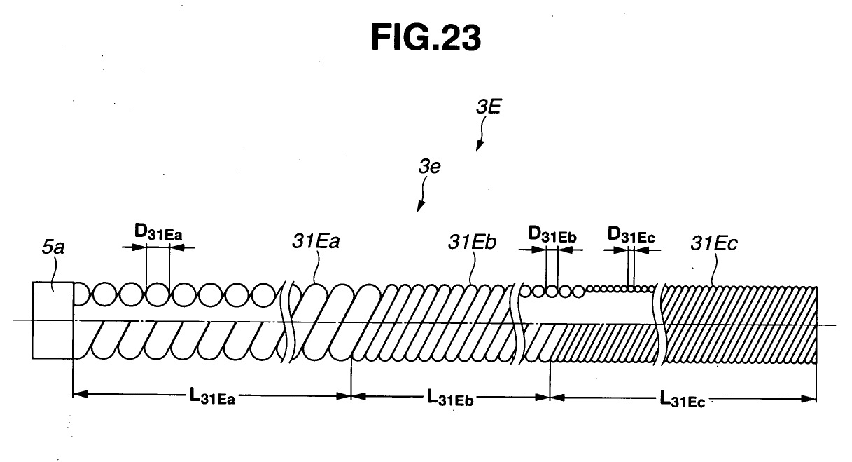

FIGS. 22 and 23 are views of an endoscope insertion portion and an endoscope system according to the present invention. FIG. 22 is an external view of an introductory tube (guide tube) configuring an endoscope system in a third embodiment and FIG. 23 is an external view of the introductory tube (guide tube) showing a deformed example of FIG. 22.

The second embodiment is configured so that the spiral-shaped portions 3a are linked with connection elements 30 at every predetermined interval as propulsive force reduction means, while the present embodiment is configured by using the spiral-shaped portions themselves as propulsive force reduction means. Other configurations are the same as for the second embodiment, therefore descriptions thereof are omitted and the same configurations have the same reference characters for description.

As shown in FIG. 22, an introductory tube (guide tube) 20D constituting an endoscope system in the third embodiment is configured so that propulsive force reduction means is disposed on the proximal end side of the spiral-shaped portion 3d by changing pitches of the spiral-shaped portions 3d at every predetermined interval toward the proximal end side as the propulsive force reduction means.

More specifically, the spiral-shaped portion 3d is constituted of: a spiral-shaped portion 31Da, a spiral-shaped portion 31Db and a spiral-shaped portion 31Dc, and is configured by winding flat metal strands formed, for example, so that a strand diameter of the spiral-shaped portion 31Db is approx. 3.5 times and a strand diameter of the spiral-shaped portion 31Dc is approx. 7 times as wide as a strand diameter D31Da of the spiral-shaped portion D31Da. In other words, the spiral-shaped portion 3d is configured so that a pitch P31Db of the spiral-shaped portion 31Db is approx. 3.5 times and a pitch P31Dc of the spiral-shaped portion 31Dc is approx. 7 times as large as a pitch P31Da of the spiral-shaped portion 31Da and has a pitch relationship: P31Da<P31Db<P31Dc.

A length L31Da of the spiral-shaped portion 31Da is, for example, a total length of a length L31a of the spiral-shaped portion 31 a and a length L30a of the connection element 30a described in the second embodiment. A length L31Db of the spiral-shaped portion 31Db is set so as to be twice as large as a length L31a of the spiral-shaped portion 31a and a length L31Dc of the spiral-shaped portion 31Dc is set so as to be four times as large as a length L31a of the spiral-shaped portion 31a. Moreover, for the spiral-shaped portion 31Da, the spiral-shaped portion 31Db and the spiral-shaped portion 31Dc, the pitches and pitch angles PA except strand diameter are all set so as to be the same.

This permits the spiral-shaped portion 3d to have high frictional force per one turn and high propulsion trigger because the pitch P31Da of the spiral-shaped portion 31Da on distal end side is smaller than the pitch P31Db of the spiral-shaped portion 31Db or the pitch P31Dc of the spiral-shaped portion 31Dc.

Accordingly, the spiral-shaped portion 3d easily provides a propulsive force on distal end having the spiral-shaped portion 31Da, but causes lower frictional force per one turn and lower propulsive force because the pitch becomes larger as the spiral-shaped portion 3d gets nearer to proximal end side as the spiral-shaped portions 31Db, 31Dc. Accordingly, the introductory tube (guide tube) causes lower propulsive force as an insertion length into the body cavity is longer. Thus, the endoscope system in the third embodiment provides the same effect as in the second embodiment.

The introductory tube (guide tube) may be configured so that a depth of the spiral groove of the spiral-shaped portion becomes smaller gradually toward the proximal end side as propulsive force reduction means.

As shown in FIG. 23, the introductory tube (guide tube) 20E is provided with the propulsive force reduction means on the proximal end side of the spiral-shaped portion 3e so that a depth of the spiral groove of the spiral-shaped portion 3e becomes smaller gradually at every predetermined interval toward the proximal end side as propulsive force reduction means.

More specifically, the spiral-shaped portion 3e is constituted of: a spiral-shaped portion 31Ea, a spiral-shaped portion 31Eb and a spiral-shaped portion 31Ec, and is configured by winding metal strands having small diameter formed, for example, so that a strand diameter D31Ec of the spiral-shaped portion 31Ec is approx. 3.5 times and a strand diameter D31Ea of the spiral-shaped portion 31Ea is approx. 7 times as large as a strand diameter D31Eb of the spiral-shaped portion D31Eb.

In other words, the spiral-shaped portion 3e is set so that a depth of the spiral groove of the spiral-shaped portion 31Ea is larger than those of the spiral grooves of the spiral-shaped portions 31Eb, 31Ec and a depth of the spiral groove of the spiral-shaped portion 31Eb is larger than that of the spiral groove of the spiral-shaped portion 31Ec.

A length L31Ea of the spiral-shaped portion 31Ea is, for example, a total length of a length L31a of the spiral-shaped portion 31a and a length L30a of the connection element 30a described in the second embodiment. A length L31Eb of the spiral-shaped portion 31Eb is set so as to be twice as large as a length L31a of the spiral-shaped portion 31a and a length L31Ec of the spiral-shaped portion 31Ec is set so as to be four times as large as a length L31a of the spiral-shaped portion 31a.

The spiral-shaped portion 3d is set so that a pitch P31Eb of the spiral-shaped portion 31Eb is approx. 3.5 times and a pitch P31Ec of the spiral-shaped portion 31Ec is set so as to be approx. 7 times as large as a pitch P31Ea of the spiral-shaped portion 31Ea. Moreover, for the spiral-shaped portion 31Ea, the spiral-shaped portion 31Eb and the spiral-shaped portion 31Ec, the pitches and pitch angles PA except strand diameter are all set so as to be the same.

Hence, in the spiral-shaped portion 3e, the spiral-shaped portion 31Ea on the distal end side is formed larger in diameter than the spiral-shaped portions 31Eb, 31Ec, so that a depth of the spiral groove of the spiral-shaped portion 31Ea is larger than those of the spiral-shaped portions 31Eb, 31Ec although the pitch P31Ea of the spiral-shaped portion 31Ea is larger than the pitch P31Eb of the spiral-shaped portion 31Eb and the pitch P31Ec of the spiral-shaped portion 31Ec. Accordingly, the spiral-shaped portion 3e provides high frictional force per one turn and high propulsion trigger.

Thus, the spiral-shaped portion 3e easily provides a propulsive force on distal end having the spiral-shaped portion 31Ea, but causes lower frictional force per one turn and lower propulsive force because the depth of the spiral groove becomes smaller as the spiral-shaped portion 3e gets nearer to the proximal end side as the spiral-shaped portions 31Eb, 31Ec. Accordingly, the introductory tube (guide tube) 20E permits lower propulsion force as an insertion length into a body cavity is longer, thus providing the same effect as in the second embodiment.

The present invention is not limited to above-described embodiments. Various changes and modifications may be made in the present invention without departing from the spirit and scope thereof.

1. An endoscope insertion portion comprising: an insertion portion capable of being inserted into a subject; a propulsive force generation section mounted on the outer periphery side of the insertion portion and rotating around the longitudinal axis of the insertion portion; and frictional force reduction means mounted on the propulsive force generation section and reducing a frictional force generated between the propulsion force generation section and a body-cavity inner wall.

2. The endoscope insertion portion according to claim 1, wherein the propulsive force generation section is constituted of a spiral-shaped portion.

3. The endoscope insertion portion according to claim 2, wherein the frictional force reduction means is a spiral portion mounted on an outer-peripheral surface of the spiral-shaped portion.

4. The endoscope insertion portion according to claim 2, wherein the frictional force reduction means is mounted on the proximal end side of the spiral-shaped portion.

5. The endoscope insertion portion according to claim 3, wherein the spiral portion is constituted by spirally winding flexible strand thinner than strand constituting the spiral-shaped portion and a distance connecting centers of spirals with each other is longer than a distance formed by connecting centers of spirals constituting the spiral-formed portion.

6. An endoscope insertion portion comprising: an insertion portion capable of being inserted into a subject; a propulsive force generation section mounted on the outer periphery side of the insertion portion and rotating around the longitudinal axis of the insertion portion; and propulsive force reduction means mounted on the propulsive force generation section and reducing a propulsive force generated by the propulsive force generation section.

7. The endoscope insertion portion according to claim 6, wherein the propulsive force generation section is constituted of a spiral-shaped portion.

8. The endoscope insertion portion according to claim 6, wherein the propulsive force reduction means is mounted on the proximal end side of the spiral-shaped portion.

9. The endoscope insertion portion according to claim 6, wherein the propulsive force reduction means is a non-spiral-shaped portion mounted on an outer-peripheral surface of the spiral-shaped portion.

10. The endoscope insertion portion according to claim 6, wherein the propulsive force reduction means is constituted so as to have fewer spiral-shaped portions toward the proximal end side of the insertion portion.

11. The endoscope insertion portion according to claim 6, wherein the propulsive force reduction means is configured so as to have a larger distance formed by connecting centers of spirals constituting the spiral-formed portion with each other toward the proximal end side of the insertion portion.

12. The endoscope insertion portion according to claim 6, wherein the propulsive force reduction means is configured so as to have a larger diameter of the strand constituting the spiral-formed portion toward the proximal end side of the insertion portion.

13. An endoscope system comprising: the endoscope insertion portion according to claim 1; a rotation device rotating the propulsive force generation section of the endoscope insertion portion around the longitudinal axis in a predetermined direction.

14. An endoscope system comprising: a slender and flexible insertion portion; a flexible insertion-portion guide portion mounted on the outer periphery side of the insertion portion and formed with, on an outer-periphery surface, a spiral-shaped portion generating a propulsive force by rotation in contact with a body-cavity inner wall; a guide portion rotation device rotating the insertion-portion guide portion around the longitudinal axis in a predetermined direction; and frictional force reduction means mounted on the insertion-portion guide portion for reducing a frictional force against the body-cavity inner wall.

15. The endoscope system according to claim 14, wherein the frictional force reduction means is configured by spirally winding flexible strand thinner than strand constituting the spiral-shaped portion of the insertion-portion guide portion.

16. The endoscope system according to claim 14, wherein the frictional force reduction means is a spiral portion mounted on an outer-peripheral surface of the spiral-shaped portion.

17. The endoscope system according to claim 14, wherein the frictional force reduction means is mounted on the proximal end side of the spiral-shaped portion.

18. The endoscope system according to claim 16, wherein the spiral portion is constituted by spirally winding flexible strand thinner than strand constituting the spiral-shaped portion and a distance connecting centers of spirals with each other is longer than a distance connecting centers of spirals constituting the spiral-formed portion.

19. An endoscope system comprising: a slender and flexible insertion portion; a flexible insertion-portion guide portion mounted on the outer periphery side of the insertion portion and formed with, on an outer-periphery surface, a spiral-shaped portion generating a propulsive force by rotation in contact with a body-cavity inner wall; a guide portion rotation device rotating the insertion-portion guide portion around the longitudinal axis in a predetermined direction; and propulsive force reduction means mounted on the insertion-portion guide portion for reducing a propulsive force of the insertion-portion guide portion.

20. The endoscope system according to claim 19, wherein the propulsive force reduction means is a non-spiral-shaped portion mounted on the insertion-portion guide portion.

21. The endoscope system according to claim 19, wherein the propulsive force reduction means is mounted on the proximal end side of the spiral-shaped portion.

22. The endoscope system according to claim 19, wherein the propulsive force reduction means is constituted so as to have fewer spiral-shaped portions toward the proximal end side of the insertion-portion guide portion.

23. The endoscope system according to claim 19, wherein the propulsive force reduction means is configured so as to have a larger distance formed by connecting centers of spirals constituting the spiral-formed portion with each other toward the proximal end side of the insertion-portion guide portion.

24. The endoscope system according to claim 19, wherein the propulsive force reduction means is configured so as to have a larger diameter of the strand constituting the spiral-formed portion toward the proximal end side of the insertion-portion guide portion.
